(19) United States
(12) Patent Application Publication
(10) Pub. No.: US 2025/0230277 A1
(43) Pub. Date: Jul. 17, 2025

(54) BIOLOGICAL FUNCTION COMPOSITE POROUS POLYESTER MICROSPHERES AND PREPARATION METHOD THEREFOR

(71) Applicant: Linghui CHEN, Changchun, Jilin (CN)

(72) Inventor: Linghui CHEN, Changchun, Jilin (CN)

(21) Appl. No.: 18/848,540

(22) PCT Filed: Mar. 21, 2023
(86) PCT No.: PCT/CN2023/082665

§ 371 Date: Sep. 19, 2024

(30) Foreign Application Priority Data
Mar. 22, 2022 (CN) 202210283743.9

(51) Int. Cl. C08G 63/91; A61K 9/50; B01J 13/02; C08J 3/16

(52) U.S. Cl. CPC C08G 63/912; A61K 9/5031; B01J 13/02; C08J 3/16; C08J 2367/04; C08J 2467/04

(57) ABSTRACT

The present invention provides biological function composite porous polyester microspheres, the polyester forming the polyester microspheres being a mixture of polylactic acid (PLA) and poly (lactic-co-glycolic acid) (PLGA); the PLGA comprises one or more of PLGA, end-group modified PLGA and bonded PLGA; the end-group modified PLGA is amino or carboxyl modified PLGA; and the bonded PLGA is end-group modified PLGA which undergoes an amino or carboxyl chemical bonding method to become loaded with one or more of hyaluronic acid (HA), collagen (Col) and cytokines. The microspheres prepared in the present invention are round in shape, have a uniform particle size distribution, have high and adjustable porosity, and simultaneously have high drug loading capacity and encapsulation efficiency.

This application claims the priority of Chinese Patent Application No. 202210283743.9, filed with the China National Intellectual Property Administration on Mar. 22, 2022, and titled with “BIOLOGICAL FUNCTION COMPOSITE POROUS POLYESTER MICROSPHERES AND PREPARATION METHOD THEREFOR”, which is hereby incorporated by reference in entirety.

### FIELD

The present application relates to the technical field of biological materials, in particular to a biological functional composite porous polyester microspheres and a preparation method therefor.

### BACKGROUND

For a long time, in order to maintain the healthy state of the skin and delay the aging of the skin, people usually adopt moisturizing and sun protection as the main treatment methods, and also adjust the skin through exercise and diet. But these methods are slow to take effect and have little effect. To quickly correct the face or eliminate wrinkles to restore the youthful state of the skin, these methods are obviously not effective. At present, facial fillers can be used to improve facial soft tissue defects, static wrinkles on the skin and tissue contours. Ideal facial fillers need to have good biocompatibility, safety and excellent cosmetic effects. In clinical practice, different types of polymer fillers are selected according to their characteristics to achieve the ideal cosmetic effect.

Among a wide variety of facial fillers, polymer fillers account for a very large proportion. Due to their advantages of good biocompatibility, biodegradability, low cost and easy modification, slight inflammation at the initial stage of injection can be avoided by physical and chemical modification in the future, for instance by introducing hydrophilic groups and active functional groups into the main chain, side chain or end group of poly-L-lactic acid (PLLA), while retaining the original biodegradability, increasing its safety and further improving the facial cosmetic effect. Polylactic acid (PLA) and poly(ε-caprolactone) (PCL) are used as raw materials of skin fillers, and many products containing PLA and/or PCL have been applied to the filling of skin wrinkles and depressions. Their main forms are PLA particles, PLA microspheres and PCL microspheres. As there are a large number of pores distributed on the surface of the microspheres and the internal pore structure is interconnected, the particle size is adjustable and the application form is free. Microspheres can not only be modified by main chain, side chain or end group, but also be combined with other functional components such as collagen, hyaluronic acid or silica gel to give them more diverse functions. However, due to different molecular structures, the degradation time after injection into the body is different. It is difficult for microspheres prepared by existing technologies to gradually control the time of functional action in terms of skin filling.

In addition, microsphere cell microcarriers can not only amplify cells in large quantities in vitro, but also serve as carriers for cells and drugs. Delivering cells or drugs to the defect site by injection has been used for bone defect repair, cartilage regeneration and myocardial repair. However, porous microsphere cell microcarriers often lack biological activity, and the material itself has no biological activity to promote cell proliferation, migration and differentiation during cell expansion in vitro or cell transmission in vivo. Obtaining biological activity by adding growth factors and other methods often has the disadvantages such as fragile growth factors, easy inactivation and high price and so on. Therefore, there is an urgent need for a microcarrier with good biological activity in tissue repair.

At present, the commonly used PLA materials have good biocompatibility and biodegradability, and have been widely used in clinic. However, the degradation products are acidic, which can easily cause decrease of local pH value and aseptic inflammatory reaction in vivo. At present, FDA has approved a variety of pharmaceutical preparations based on PLA microspheres to enter clinical practice. However, at present, most of the injected microspheres have single structural characteristics and biological characteristics, which cannot meet the current needs.

### SUMMARY

In view of this, the technical problem to be solved by the present application is to provide a biological function composite porous polyester microspheres and a preparation method therefor, which can realize the function of gradually releasing bioactive molecules in an early stage, middle stage and later stage.

The present application provides a biological functional composite porous polyester microspheres, wherein the polyester forming the polyester microspheres is a mixture of PLA and PLGA;

The PLGA comprises one or more of PLGA, end-group modified PLGA and bonded PLGA;

The end-group modified PLGA is amino or carboxyl modified PLGA;
the bonded PLGA is end-group modified PLGA loaded with one or more of HA, Col and cytokines by chemical bonding with amino or carboxyl groups.

In the present application, the PLA refers to polylactic acid, and the PLGA refers to poly (lactic-co-glycolic acid).

In the present application, preferably, a mass ratio of the PLA and the PLGA is 50:0 to 0:50, and preferably the mass ratio is not 0, further preferably 49:1 to 1:49, more preferably 90:10 to 10:90, specifically can be 90:10, 70:30, 10:10, 30:70 or 10:90.

In the present application, preferably, the PLA is one or more of D type, L type and DL type.

In the present application, preferably, a ratio of LA and GA in the PLGA is 10:90 to 90:10, more preferably one of 75:25, 50:50 and 25:75.

In the present application, preferably, the PLA has a molecular weight of 2000 Da to 100000 Da.

In the present application, preferably, the PLGA has a molecular weight of 2000 Da to 100000 Da.

In the present application, preferably, the amino-modified PLGA or carboxyl-modified PLGA, namely PLGA-NH₂ or PLGA-COOH, has a molecular weight of 2000 Da to 100000 Da.

The bonded PLGA is end-group modified PLGA loaded with one or more bioactive factors such as HA, Col and cytokines by chemical bonding with amino or carboxyl groups.

In the present application, when the PLGA is an end-group modified PLGA, the polyester microspheres can also be loaded with one or more bioactive factors such as HA, Col and cytokines. The above-mentioned loading can be carried out by means of chemical bonding with amino or carboxyl groups.

In the present application, the HA refers to hyaluronic acid; Col refers to collagen.

The particle size of the above-mentioned biological function composite porous polyester microspheres prepared by the present application is 20 μm to 800 μm.

According to the present application, a double emulsion-solvent evaporation method is adopted, blank microspheres are prepared by using PLA with different chirality and PLGA or PLGA with end group modification (PLGA-COOH, PLGA-NH₂); bonded microspheres are prepared by end group bonding (PLGA-HA, PLGA-Col); the blank microspheres are loaded with biological functional substances to prepare composite polyester microspheres, composite injection porous microspheres are prepared by different polyester materials, and degradation time, particle size, microporous size and distribution can be adjusted as desired; functional substances such as hyaluronic acid or gelatin are compounded with porous microspheres to prepare functional composite polyester microspheres, which are injected into the body to play a variety of biological functions. The experimental results show that the results of promoting collagen production are: blank microspheres <composite microspheres <bonded microspheres.

The present application provides a method for preparing the biological functional composite porous polyester microspheres, which comprises the following steps:
S1) dissolving a polyester compound in an organic solvent to obtain an organic phase; wherein the polyester compound comprises PLA and PLGA;
dissolving NH₄CO₃ in deionized water to obtain water phase;
S2) adding the water phase into the organic phase, and emulsifying at 500 rpm to 50000 rpm to obtain a primary emulsion;
S3) adding the primary emulsion into the polyvinyl alcohol solution, and emulsifying at 50 rpm to 5000 rpm to obtain the biological function composite porous polyester microspheres.

In the present application, preferably, the organic solvent is one or more of dichloromethane, chloroform, acetone, ethyl acetate and benzyl alcohol.

In the present application, preferably, a mass content of the polyester compound in the organic phase is 2 mg/mL to 500 mg/mL.

In the present application, preferably, a mass content of NH₄CO₃ in the water phase is 0.1% to 20% (W/V), more preferably 2% to 10% (W/V), specifically can be 1% (W/V), 5% (W/V) or 10% (W/V).

In the present application, preferably, a concentration of the polyvinyl alcohol solution is 1% % to 500% % (W/V), more preferably 5% % to 20% o.

In the present application, preferably, the emulsifying rotation speed in the above-mentioned step S2) is 3000 rpm to 8000 rpm.

In the present application, preferably, the above-mentioned step S2) is carried out in an ice bath.

In the present application, preferably, the emulsifying rotation speed in above-mentioned step S3) is 100 rpm to 800 rpm.

Compared with the prior art, the present application provides a biological functional composite porous polyester microspheres, wherein the polyester forming the polyester microsphere is a copolymer of PLA and PLGA; the PLGA is PLGA, end-group modified PLGA or bonded PLGA; the end-group modified PLGA is amino or carboxyl modified PLGA; the bonded PLGA is end-group modified PLGA loaded with one or more of HA, Col and cytokines by chemical bonding with amino or carboxyl groups.

The microspheres prepared in the present application are round in shape, have uniform size distribution, and have high drug loading capacity and high encapsulation efficiency, wherein the drug loading capacity can reach more than 49.2% and the encapsulation efficiency can reach more than 99.0%. It can realize the gradual release function of bioactive molecules in an early stage, middle stage and later stage, that is, by adjusting the mass ratios of PLA and PLGA in the polyester microspheres, the degradation time thereof in the body is adjusted, for instance to 2 months, 6 months, 24 months, thus bringing about biological effects at different times.

In the present application, PLA porous microspheres are prepared by PLA with different chirality, the polyester end groups are modified with carboxyl and amino, and the end groups are bonded with hyaluronic acid and collagen, to prepare modified porous polyester microspheres and bonded porous polyester microspheres, and the size of the microspheres can be adjusted by changing the emulsifying rotation speed; the size and distribution of micropores can be adjusted by changing the dosage of pore-forming agent. In addition, the functional substances are compounded with the porous microspheres to prepare functional composite microspheres, which are injected into the body to play a variety of biological functions.

### BRIEF DESCRIPTION OF DRAWINGS

FIG. 1 is the SEM image of microspheres prepared by 1% NH₄CO₃ in Example 1;

FIG. 2 is the SEM image of microspheres prepared by 10% NH₄CO₃ in Example 1;

FIG. 3 is the nuclear magnetic hydrogen spectrum of PGLA-COOH in example 2;

FIG. 4 is the nuclear magnetic hydrogen spectrum of PGLA-NH₂ in Example 3;

FIG. 5 is an infrared absorption spectrum of PGLA-HA in example 4;

FIG. 6 is an infrared absorption spectrum of PGLA-Col in example 5;

FIG. 7 show that effects of different microspheres in example 8 on promoting collagen production.

### DETAILED DESCRIPTION

In order to further illustrate the present application, the biological function polyester microspheres and the preparation method therefor provided by present application are described in detail below with reference to the embodiments. All other embodiments obtained by those skilled in the art based on the embodiments in the present application without any creative work belong to the scope of protection of the present application.

### Example 1

### Preparation Method of Composite Porous Polyester Microspheres:

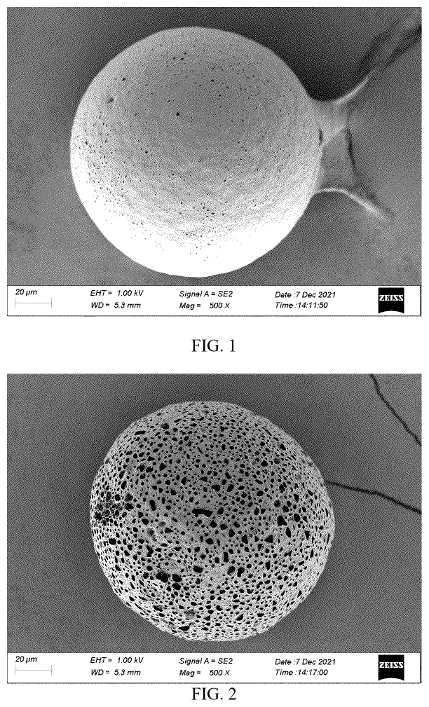

(a) Preparation of organic phase: 500 mg of polyester compound was weighed and dissolved in 8 mL of dichloromethane, and stirred evenly, wherein in the polyester compound, PLA and PLGA were in a ratio of 1:1 and in the PLGA, LA and GA were a ratio of 75:25;
(b) Preparation of water phase: NH₄CO₃ was weighed and dissolved in deionized water, wherein a content (wt %) of NH₄CO₃ was 1% and 10% respectively;
(c) The two water phases were added into the organic phase and emulsified in an ice bath at 5000 rpm for 3 min to obtain a primary emulsion, wherein the rotation speed could be 5000 rpm;
(d) The primary emulsion was immediately poured into a beaker containing 300 ml of 0.1% (W/V) concentration of polyvinyl alcohol (PVA) and emulsified at 400 rpm for 4 h, wherein the rotation speed could be 400 rpm; the emulsification time could be 4 h;
(e) The microspheres were collected by static or centrifugation and washed with distilled water for three times, and then freeze-dried to obtain two kinds of composite porous polyester microspheres with different pore sizes (FIGS. 1 and 2).

### Example 2

Preparation method of modified PLGA-COOH: PLGA, 4-(dimethylamino)pyridine, succinic anhydride and dichloromethane were mixed in a mass ratio of 5:1:1:50, fully dissolved and stirred for 4 h, distilled under reduced pressure, then precipitated in methanol for 3 times, and vacuum dried to obtain PLGA-COOH. As shown in FIG. 1, PLGA-COOH was successfully prepared.

### Example 3

Preparation method of modified PLGA-NH₂: PLGA-COOH was dissolved in anhydrous dichloromethane, and N,N′-carbonyldiimidazole (CDI) was added to the solution, and the mixture was activated for 1 h, added with hexylenediamine, and reacted for 12 h, the reactant was precipitated with ethanol for 3 times, and dried in vacuum to obtain the PLGA-NH₂. As shown in FIG. 2, PLGA-NH₂ was successfully prepared.

### Example 4

PLGA-HA was obtained by conventional operable condensation reaction. Specifically, HA was dissolved in 3 mL of water to obtain a HA solution with a concentration of 0.5 g/ml, and then 1.55 g of 1-ethyl-(3-dimethylaminopropyl) carbodiimide (EDC) and 1.15 g of N-hydroxysuccinimide (NHS) were added. The mixture was fully stirred and reacted for 30 min to activate carboxyl groups in HA. 1.25 g of PLGA-NH₂ was dissolved in 3 ml of N, N-dimethylformamide (DMF) solution, and then the obtained solution was added dropwise into the above-mentioned HA solution, stirred and reacted for 24 h, and then dialyzed and freeze-dried to obtain PLGA-HA polyester material. The nuclear magnetic resonance hydrogen spectrum and

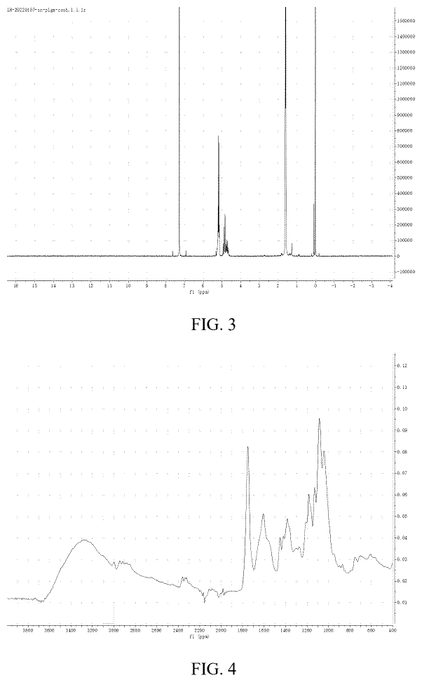

Fourier infrared spectra are shown in FIGS. 3 and 4, respectively. Meanwhile, HA-bonded composite polyester microspheres could be prepared according to the scheme of Example 1, in which the content of NH₄CO₃ (wt %) was 10%.

### Example 5

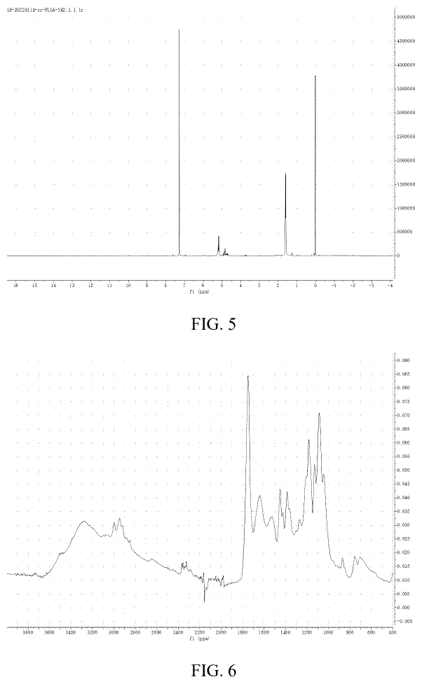

PLGA-Col was obtained by conventional operable condensation reaction. Specifically, PLGA-COOH was dissolved in 3 mL of DMF to obtain a solution of PLGA-COOH with a concentration of 1 g/ml, then 1.55 g of EDC and 1.15 g of NHS were added. The mixture was fully stirred for 30 min, and precipitated with ether to obtain PLGA-NHS. 1.25 g of PLGA-NHS was dissolved in 3 mL of DMF solution, and then the obtained solution was added dropwise into 3 ml of Col aqueous solution with a concentration of 0.25 g/mL, and stirred and reacted for 24 h, to obtain the PLGA-Col polyester material. The nuclear magnetic resonance hydrogen spectrum and Fourier infrared spectra are shown in FIGS. 5 and 6, respectively.

### Example 6

50 mg of the microspheres with large pore size obtained in Example 1 (FIG. 2) and 50 mg of HA were dissolved in 2 mL of distilled water for ultrasonic compounding, and the obtained product was washed with water to obtain the HA-loaded microspheres. The HA loading rate and encapsulation rate of the microspheres obtained by present application are 49.2% and 99.0% respectively.

### Example 7

The PLGA-HA obtained in Example 4 was mixed with PLA in a mass ratios of 90:10, 70:30, 50:50, 30:70 and 10:90 respectively, and then five kinds of different HA-bonded polyester microspheres were prepared according to the method of Example 1. The obtained polyester microspheres were subjected to particle size detection by a particle size meter; the obtained polyester microspheres were placed in phosphate buffer solution (PBS), and then the solution was sampled at different time points. The stability and long-term effect of the microspheres were verified by detecting the concentration of HA in the solution, wherein the total amount of HA bonded in the polyester microspheres was set as 100%. As shown in Table 1, the polyester microspheres have a uniform particle size distribution and a small dispersion index (PDI). As shown in Table 2, the polyester microspheres have good stability and long-term effect.

TABLE 1
Particle size and PDI of different
HA-bonded polyester microspheres

| | Average particle size | |
|---|---|---|
| Microspheres | (μm) | PDI |
| 90:10 | 50.3 | 0.13 |
| 70:30 | 49.6 | 0.16 |
| 50:50 | 51.0 | 0.11 |
| 30:70 | 50.4 | 0.14 |
| 1:9 | 51.7 | 0.12 |

TABLE 2
HA release of HA-bonded polyester microspheres at different times

| | 7 days | 15 days | 30 days | 45 days | 60 days |
|---|---|---|---|---|---|
| | release | release | release | release | release |
| Microspheres | rate (%) | rate (%) | rate (%) | rate (%) | rate (%) |
| 90:10 | 6.23 | 8.34 | 10.45 | 13.54 | 20.46 |
| 70:30 | 5.11 | 7.28 | 9.28 | 11.22 | 16.44 |
| 50:50 | 3.88 | 4.28 | 6.28 | 8.23 | 12.45 |
| 30:70 | 2.55 | 3.11 | 4.15 | 6.94 | 9.55 |
| 10:90 | 1.53 | 2.12 | 3.17 | 5.27 | 8.34 |

### Example 8

The PLGA-Col obtained in Example 5 was mixed with PLA in a mass ratios of 90:10, 70:30, 50:50, 30:70 and 10:90 respectively, and then five kinds of different Col-bonded polyester microspheres were prepared according to the method of Example 1. The obtained polyester microspheres were subjected to particle size detection by a particle size meter; the obtained polyester microspheres were placed in PBS, and then the solution was sampled at different time points. The stability and long-term effect of the microspheres were verified by detecting the concentration of Col in the solution, wherein the total amount of Col bonded in the polyester microspheres was set as 100%. As shown in Table 3, the polyester microspheres have a uniform particle size distribution and a small PDI. As shown in Table 4, the polyester microspheres have good stability and long-term effect.

TABLE 3
Particle size and PDI of different
Col-bonded polyester microspheres

| | Average particle size | |
|---|---|---|
| Microspheres | (μm) | PDI |
| 90:10 | 51.2 | 0.11 |
| 70:30 | 50.3 | 0.15 |
| 50:50 | 51.6 | 0.13 |
| 30:70 | 50.9 | 0.10 |
| 10:90 | 48.7 | 0.09 |

TABLE 4
Col release of Col-bonded polyester microspheres at different times

| | 7 days | 15 days | 30 days | 45 days | 60 days |
|---|---|---|---|---|---|
| | release | release | release | release | release |
| Microspheres | rate (%) | rate (%) | rate (%) | rate (%) | rate (%) |
| 90:10 | 6.55 | 9.11 | 11.23 | 15.66 | 16.44 |
| 70:30 | 5.11 | 6.22 | 7.34 | 9.66 | 10.34 |
| 50:50 | 4.43 | 5.33 | 6.45 | 7.35 | 9.56 |
| 30:70 | 3.13 | 4.45 | 5.34 | 6.29 | 7.94 |
| 10:90 | 1.11 | 2.18 | 3.38 | 4.77 | 5.13 |

### Example 9

According to the experimental scheme of Example 1, only the preparation method of organic phase was adjusted. 500 mg of polyester compound was weighed and dissolved in 8 mL of dichloromethane, respectively, and the mixture was stirred evenly, wherein the ratio of the polyester compounds PLA and PLGA was 90:10, 70:30, 50:50, 30:70 and 10:90 respectively, and the ratio of LA and GA in PLGA was 50:50. The content of NH₄CO₃ (wt %) was 5% during the preparation of microspheres. The three kinds of prepared microspheres were placed in PBS to study their degradation performance in vitro respectively, and the initial mass of microspheres was set as 100%. As shown in Table 5, the degradation time of polyester microspheres could be adjusted by changing the ratio of PLA and PLGA, the degradation time could be 2 months to 6 months and even more than 24 months.

TABLE 5
Degradation of polyester microspheres
with different ratios of PLA and PLGA

| | 2 | 6 | 12 | 18 | 24 |
|---|---|---|---|---|---|
| | months | months | months | months | months |
| | remaining | remaining | remaining | remaining | remaining |
| Micro- | quantity | quantity | quantity | quantity | quantity |
| spheres | (%) | (%) | (%) | (%) | (%) |
| 90:10 | 90.77 | 72.66 | 58.23 | 35.66 | 19.77 |
| 70:30 | 78.92 | 52.55 | 16.66 | — | — |
| 50:50 | 70.43 | 14.22 | — | — | — |
| 30:70 | 23.13 | — | — | — | — |
| 10:90 | 1.11 | — | — | — | — |

### Example 10

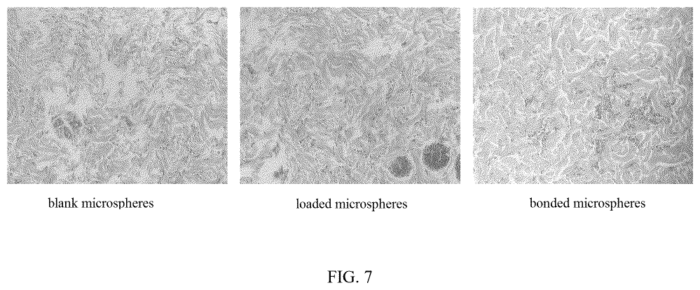

Polyester microspheres, namely, blank microspheres, loaded microspheres and bonded microspheres, were prepared according to the methods of Examples 1, Examples 6 and Examples 7 (PLA:PLGA=10:10), and then injected into mice by intradermal injection for functional verification. The collagen content was compared and analyzed. As shown in FIG. 7, the results of promoting collagen production were as follows: blank microspheres <loaded microspheres <bonded microspheres.

The above examples are described only to help understand the method and core concept of the present application. It should be noted that, for those of ordinary skill in the art, many improvements and modifications may be further made to the present application without departing from the principle of the present application, and these improvements and modifications also fall within the protection scope of claims of the present application.

The invention claimed is:

1. A biological function composite porous polyester microspheres, wherein the polyester forming the polyester microspheres is a mixture of PLA and PLGA;
the PLGA comprises one or more of PLGA, end-group modified PLGA and bonded PLGA;
the end-group modified PLGA is amino and/or carboxyl modified PLGA;
the bonded PLGA is end-group modified PLGA loaded with one or more of HA, Col and cytokines by chemical bonding with amino or carboxyl groups.

2. The biological function composite porous polyester microspheres according to claim 1, wherein a mass ratio of the PLA and the PLGA is 50:0 to 0:50.

3. The biological function composite porous polyester microspheres according to claim 1, wherein the PLA is one or more of D type, L type and DL type.

4. The biological functional composite porous polyester microspheres according to claim 1, wherein a molar ratio of lactide (LA) and glycolide (GA) in the PLGA is 10:90 to 90:10.

5. The biological function composite porous polyester microspheres according to claim 1, wherein the PLA has a molecular weight of 2000 Da to 100000 Da.

6. The biological functional composite porous polyester microspheres according to claim 1, wherein the PLGA has a molecular weight of 2000 Da to 100000 Da.

7. The biological functional composite porous polyester microspheres according to claim 1, wherein when the PLGA is an end-group modified PLGA, the polyester microsphere is also loaded with one or more of HA, Col and cytokines.

8. A method for preparing the biological functional composite porous polyester microspheres according to claim 1, comprising the following steps:
S1) dissolving a polyester compound in an organic solvent to obtain an organic phase; wherein the polyester compound comprises PLA and PLGA;
dissolving NH₄CO₃ in deionized water to obtain water phase;
S2) adding the water phase into the organic phase, and emulsifying at 500 rpm to 50000 rpm to obtain a primary emulsion;
S3) adding the primary emulsion into a polyvinyl alcohol solution, and emulsifying at 50 rpm to 5000 rpm to obtain the biological function composite porous polyester microspheres.

9. The method according to claim 8, wherein the organic solvent is one or more of dichloromethane, chloroform, acetone, ethyl acetate and benzyl alcohol.

10. The method according to claim 8, wherein a mass content of the polyester compound in the organic phase is 2 mg/mL to 500 mg/mL;
a mass content of NH₄CO₃ in the water phase is 0.1% to 20% (W/V);
a concentration of the polyvinyl alcohol solution is 1% % to 500% % (W/V).
